(19) United States
(12) Patent Application Publication
(10) Pub. No.: US 2026/0207870 A1
(43) Pub. Date: Jul. 23, 2026

(54) WEARABLE POSITIVE AIRFLOW PRESSURE APPARATUS

(71) Applicant: Elijah Joseph Paparella, Palo Alto, CA (US)

(72) Inventor: Elijah Joseph Paparella, Palo Alto, CA (US)

(21) Appl. No.: 19/328,444

(22) Filed: Sep. 15, 2025

Related U.S. Application Data
(60) Provisional application No. 63/747,054, filed on Jan. 19, 2025.

(51) Int. Cl. A61M 16/06; A61M 16/00

(52) U.S. Cl. CPC A61M 16/0616; A61M 16/0003; A61M 16/022; A61M 2016/0027; A61M 2205/8206

(57) ABSTRACT

A positive airflow pressure apparatus for generating a positive airflow pressure in a user's breathing air passage is provided. The apparatus comprises a motor housing having a housing inlet and a housing outlet, with a motor housed within the motor housing for receiving air from the housing inlet and for propelling the air to the housing outlet, for creating positive airflow pressure at the housing outlet. The apparatus further has a face mask for breathing engagement with the user's breathing air passage, the face mask attached to the motor housing proximate the housing outlet, for receiving the propelled air through the housing outlet. The motor and the face mask are for operating together to generate the positive airflow pressure in the user's breathing air passage. The apparatus further has a face mask connector for physically attaching the face mask and the motor housing to the user.

### CROSS-REFERENCE TO RELATED APPLICATIONS

This non-provisional application claims priority from U.S. Provisional Patent Application No. 63/747,054, filed Jan. 19, 2025, which is incorporated herein by reference in its entirety.

### TECHNICAL FIELD

The present invention relates to medical devices including at least sleep aides, respirators, and ventilators. More particularly, the invention relates to apparatuses and methods for applying various modes of operation, such as continuous positive air pressure (CPAP) to a user's air passages and/or anti-snoring apparatuses, among other modes of operation, such as while they sleep.

### BACKGROUND OF THE INVENTION

Currently, CPAP devices, such as for treating sleep apnea, are large, cumbersome, onerous and/or impossible to clean, difficult to travel with, contain many parts, and complicated to service. Some components, such as a hose connecting a face mask to a base unit housing a motor, controls, and other components, are recommended to be replaced every ninety days by the manufacturer, since there is no recommended method to effectively clean such components. These replacement recommendations create needless environmental waste and expense. Furthermore, current CPAP devices have separate humidity chambers requiring distilled water for humidification, and do not have locking controls preventing a user's unconscious removal of the face mask while sleeping, which is one of the more significant problems preventing the proper treatment of sleep apnea. Many CPAP device users continue to suffer from untreated sleep apnea due to these and other problems.

The present invention is provided to solve the problems discussed above and other problems, and to provide advantages and aspects not provided by prior systems and methods of this type. A full discussion of the features and advantages of the present invention is deferred to the following detailed description, which proceeds with reference to the accompanying drawings.

### SUMMARY OF THE INVENTION

One embodiment of the present invention provides an improved apparatus for applying CPAP, such as while a user sleeps, without separating the motor and control unit from the face mask. In one embodiment, a positive airflow pressure apparatus for generating a positive airflow pressure in a user's breathing air passage is provided. The apparatus can be worn by the user, and is portable for the user to be able to move around with the apparatus while wearing the apparatus and for the user to being able to carry the apparatus with the user and/or travel with the apparatus. The apparatus has a motor housing with a housing inlet and a housing outlet. The motor, which can be a variable speed motor, is housed within the motor housing for receiving air from the housing inlet and for propelling the air through the housing outlet, for creating positive airflow pressure at the housing outlet. The apparatus further has a face mask for breathing engagement with the user's air breathing passage, the face mask attached to the motor housing proximate the housing outlet, for receiving the propelled air through the housing outlet. The motor and the face mask are for operating together to generate the positive airflow pressure in the user's breathing air passage. The apparatus further has a face mask connector for physically removably attaching the face mask and the motor housing to the user, allowing the use to wear the apparatus. In one embodiment, there is no need for a hose between motor housing and the face mask, as will be described in greater detail below.

In a further embodiment of the present invention, the motor housing has a main housing portion, an end cap, and an inlet plate. The housing inlet can be formed as a part of the inlet plate. The inlet plate is connected to the main housing portion, and can be sealed thereto in order to make the housing substantially airtight. The inlet plate and an inner wall of the main housing portion define a housing front end chamber.

In a further embodiment, the apparatus further has an air restrictor plate secured in the housing front end chamber for separating the housing front end chamber into a first front end chamber on a first side of the air restrictor plate and a second front end chamber on a second side of the air restrictor plate. The air restrictor plate has a plurality of restrictor air holes for allowing air to pass from the first front end chamber to the second front end chamber, and vice versa. The apparatus also has a differential pressure sensor positioned between the first front end chamber and the second front end chamber for sensing a pressure differential between the first front end chamber and the second front end chamber as air flows between the first and second front end chambers. The apparatus further has an air pressure sensor for sensing an air pressure proximate the housing outlet.

In a further embodiment, the apparatus has an onboard controller which receives a pressure differential signal representing the pressure differential between the first front end chamber and the second front end chamber. The onboard controller also receives an air pressure signal representing the air pressure proximate the housing outlet. The onboard controller continuously determines and controls the speed of the variable speed motor form of the motor in response to the sensed air pressure and the sensed pressure differential, for generating the positive airflow pressure in the user's air passage.

In one embodiment, the apparatus can be powered by a rechargeable battery connected to and powering the onboard controller and/or the power/control apparatus and motor. A charger can be removably attached to an rechargeable battery to recharge the rechargeable battery when the battery charge becomes low. In another embodiment, the apparatus has a wired power module connected to and powering the onboard controller and motor. The apparatus can be powered with the rechargeable battery included in a “pass through charging configuration” while also plugged into the wall.

In a further embodiment, the apparatus has a first sealing gasket and second sealing gasket. The first sealing gasket circumferentially surrounds an outlet portion of the motor and the second sealing gasket circumferentially surrounds an inlet portion of the motor. The first sealing gasket and the second sealing gasket can abut one another, or be positioned next to or adjacent one another, proximate a central portion of the motor. The first and second sealing gaskets are provided for at least reducing any motor noise to the outside of the motor housing when the motor is being operated and/or for sealing airflow and ensuring all air is taken in through the housing inlet and propelled through the housing outlet with no or minimal air leakage.

In one embodiment, the face mask has a main mask portion for covering the nose air passage of a user or the mouth air passage of a user, or both air passages. The face mask can further have a connection receiver for receiving and attaching the face mask connector to the face mask, for physically attaching the face mask and the motor housing to the user. The connection receiver can have a first connection receiver for receiving and removably attaching a left portion of a bottom connector of the face mask connector to the face mask. The connection receiver can also have a second connection receiver for receiving and removably attaching a right portion of the bottom connector of the face mask connector to the face mask. The connection receiver can further have a third connection receiver for receiving and attaching a top connector of the face mask connector to the face mask.

In one embodiment, the first connection receiver is attached to a left side of the main mask portion, the second connection receiver is attached to a right side of the main mask portion, and the third connection receiver is attached to a front top portion of the main mask portion via a support. In a further embodiment, the third connection receiver can have a T-joint extending from the support, a first arm extending from the left side of the T-joint, and a second arm extending from the right side of the T-joint. In this embodiment, the first arm is for receiving and removably attaching a left portion of a top connector of the face mask connector to the face mask, and the second arm is for receiving and removably attaching a right portion of the top connector of the face mask connector to the face mask.

In one embodiment of the apparatus, the main mask portion of the face mask further has air holes for allowing air to exit the face mask when the user exhales. The face mask can also have an air treatment chamber side wall extending from a front portion of the face mask, and an air treatment chamber front wall extending from the air treatment chamber side wall to create an air treatment chamber for receiving an air treatment device. The air treatment device can preferably be a dehumidification device or a humidification device, for reducing or increasing, respectively, the humidity of the air entering the user's air passage. The air treatment device can also be an anti-bacterial filter to prevent and separate contaminated air inside the face mask chamber from entering the motor housing.

In one embodiment of the apparatus of the present invention, the face mask further has a housing connector attached to a front portion of the face mask. The housing connector can have a flat front wall and a plurality of tabs for removably connecting the face mask to the motor housing. A bore is formed through a central portion the housing connector for allowing air to flow between the housing connector and the motor housing.

In one embodiment, the face mask can have an air restrictor or limiter attached to a front portion of the face mask for allowing air to enter the face mask from the motor housing, and for preventing air from flowing back into the motor housing from the face mask. In one embodiment, the air restrictor/limiter can take the form of a one-way valve.

As mentioned, the motor housing has a main housing portion. The motor housing also has an end cap attached to the motor housing on the opposite side of the motor housing from the housing inlet. The end cap has an interior side wall establishing an air through hole or housing outlet. A plurality of flanges extend from the interior side wall for receiving a respective plurality of tabs of the face mask, and for allowing for removable attachment of the motor housing to the face mask.

In a further embodiment of the apparatus of the present invention, the face mask connector has a top connector with a top elongated portion, a left closed end loop, and a right closed end loop. The face mask connector further has a bottom connector with a bottom elongated portion, a left open end loop, and a right open end loop. The face mask connector also has a joiner for connecting the top elongated portion of the top connector to the bottom elongated portion of the bottom connector, for maintaining generally consistent distance between the top elongated portion and the bottom elongated portion. In one embodiment, the bottom connector can have a plurality of left through holes and a plurality of right through holes. The left open end loop can have a left end dowel and the right open end loop can have a right end dowel. The left end dowel can be inserted in at least one of the plurality of left through holes for securing and connecting the left open end loop to the first connection receiver of the face mask. Likewise, the right end dowel can be inserted in at least one of the plurality of right through holes for securing and connecting the right open end loop to the second connection receiver of the face mask. The left closed end loop and the right closed end loop are for securing and connecting the top connector to the third connection receiver of the face mask.

In an even further embodiment, the bottom connector can have a left hook with a left opening and a right hook with a right opening. The left open end loop can be inserted within the left opening and the right open end loop can be inserted in the right opening. The left hook is for securing and connecting the left open end loop to the first connection receiver of the face mask, and the right hook is for securing and connecting the right open end loop to the second connection receiver of the face mask.

As mentioned, an onboard controller can control the operation of the motor based on input signals from the differential pressure sensor and the air pressure sensor. The onboard controller can have memory for storing software and/or firmware, including algorithms, for performing such standard operations. The software and/or firmware can be programmable and can allow for customizations for each user's attributes and conditions, each of which may require that, and/or allow for, certain custom settings to be used for each user. These features and functionality can alternatively be provided in a non-onboard controller in addition to and/or instead of the onboard controller, with the onboard controller having limited functionality relative to the motor functions and relative to receiving signals from the differential pressure sensor and the air pressure sensor in the latter embodiment.

Other features and advantages of the invention will be apparent from the following specification taken in conjunction with the following drawings.

### BRIEF DESCRIPTION OF THE DRAWINGS

To understand the present invention, it will now be described by way of example, with reference to the accompanying drawings in which:

FIG. 1 is a perspective view of a positive airflow pressure apparatus, such as a wearable CPAP apparatus, shown being worn by a user;

FIG. 2 is right side view of the positive airflow pressure apparatus illustrated in FIG. 1 in a smaller motor housing configuration shown being worn by the user;

FIG. 3 is cut away left side view of the positive airflow pressure apparatus illustrated in FIG. 1 (cut away along a central vertical line from a front view) shown being worn by the user;

FIG. 4 is an exploded parts perspective view of the positive airflow pressure apparatus illustrated in FIG. 1 shown being worn by the user;

FIG. 5 is an exploded parts cut away left side view of the positive airflow pressure apparatus illustrated in FIG. 1 (cut away along a central vertical line from a front view) shown being worn by the user;

FIG. 6 is perspective view of one embodiment of a face mask connector or head harness of the positive airflow pressure apparatus illustrated in FIG. 1;

FIG. 7 is top view of the head harness illustrated in FIG. 6;

FIG. 8 is perspective view of another embodiment of a head harness of the positive airflow pressure apparatus illustrated in FIG. 1;

FIG. 9 is top view of the head harness illustrated in FIG. 8;

FIG. 10 is perspective view of one embodiment of a face mask of the positive airflow pressure apparatus illustrated in FIG. 1;

FIG. 11 is front view of the face mask illustrated in FIG. 10;

FIG. 12 is left side view of the face mask illustrated in FIG. 10;

FIG. 13 is rear view of the face mask illustrated in FIG. 10;

FIG. 14 is a right side view of an inlet plate of the positive airflow pressure apparatus illustrated in FIG. 1, with an inlet air filter not inserted in the inlet plate;

FIG. 15 is a front view of the inlet plate illustrated in FIG. 14, with the inlet air filter not inserted in the inlet plate;

FIG. 16 is a rear view of the inlet plate illustrated in FIG. 14, with the inlet air filter not inserted in the inlet plate;

FIG. 17 is a perspective view of a mask air humidity/dehumidity/antibacterial filter of the positive airflow pressure apparatus illustrated in FIG. 1;

FIG. 18 is a perspective view of an air restrictor/barrier plate/orifice plate of the positive airflow pressure apparatus illustrated in FIG. 1;

FIG. 19 is a left side view of the air restrictor/barrier plate illustrated in FIG. 18;

FIG. 20 is a front view of the air restrictor/barrier plate illustrated in FIG. 18, rotated 180 degrees;

FIG. 21 is a rear perspective view of an air flow or blower motor of the positive airflow pressure apparatus illustrated in FIG. 1;

FIG. 22 is rear view of the air flow motor illustrated in FIG. 21;

FIG. 23 is left side view of a first sealing gasket and a second sealing gasket of the positive airflow pressure apparatus illustrated in FIG. 1, with the first and second sealing gaskets shown adjacent one another as they would be inserted into a main housing of the positive airflow pressure apparatus illustrated in FIG. 1;

FIG. 24 is cut away view of a first sealing gasket and a second sealing gasket illustrated in FIG. 23, with the air flow motor of FIG. 21 inserted therein (cut away along a central vertical line from a front view);

FIG. 25 is a perspective view of an end cap of the positive airflow pressure apparatus illustrated in FIG. 1;

FIG. 26 is a front view of the end cap illustrated in FIG. 25, with the end cap rotated 180 degrees;

FIG. 27 is a rear view of the end cap illustrated in FIG. 25, with the end cap rotated 180 degrees;

FIG. 28 is a bottom view of the end cap illustrated in FIG. 25;

FIG. 29 is a perspective view of the power and control apparatus of the positive airflow pressure apparatus illustrated in FIG. 1;

FIG. 30 is a top view of a control board of the power and control apparatus illustrated in FIG. 29;

FIG. 31 is a left side view of the control board of the power and control apparatus illustrated in FIG. 29;

FIG. 32 is perspective view of one embodiment of a wrapping/cover/casing which is shown in a manner that wraps the motor housing of the positive airflow pressure apparatus illustrated in FIG. 1;

FIG. 33 is a right side view of the wrapping illustrated in FIG. 32;

FIG. 34 is a rear view of the wrapping illustrated in FIG. 32;

FIG. 35 is cut away perspective view of the positive airflow pressure apparatus illustrated in FIG. 1;

FIG. 36 is a flow diagram illustrating significant operation steps in the operation of the positive airflow pressure apparatus illustrated in FIG. 1, along with the respective significant components involved at each step, when the user is inhaling;

FIG. 37 is a block diagram illustrating how the control board, of the positive airflow pressure apparatus illustrated in FIG. 1; operates in conjunction with the motor and communication features at a higher level; and,

FIG. 38 is a block diagram illustrating significant components of an external control board and power module of the positive airflow pressure apparatus illustrated in FIG. 1, and how such components operate together at a higher level.

### DETAILED DESCRIPTION

While this invention is susceptible of embodiments in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated.

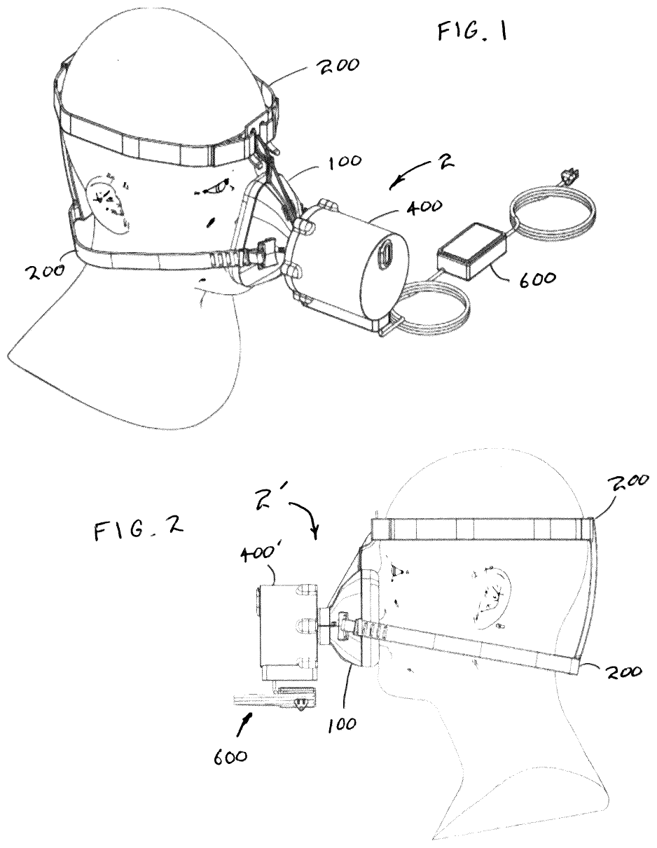

FIGS. 1 and 2 illustrate two embodiments of a positive airflow pressure apparatus 2, 2′ of the present invention with a user wearing the apparatus 2, 2′. In one embodiment, the apparatus 2, 2′ is a wearable CPAP apparatus. The apparatus 2, 2′ shown in FIGS. 1 and 2 has a motor housing 400, 400′ for housing a motor 8, which is shown and described in more detail in relation to subsequent figures. The apparatus 2, 2′ shown in FIGS. 1 and 2 further has a face mask 100 connected to the motor housing 400, 400′ for engaging with a user's face for covering at least one air passageway of the user—the nose and/or the mouth of the user. The face mask 100 shown in FIGS. 1 and 2 covers both the mouth and the nose of the user. The apparatus 2, 2′ of FIGS. 1 and 2 also has a face mask connector 200 for removably attaching the apparatus 2, 2′ to the user's head. The apparatus 2, 2′ further has power module or unit 600, 2902 for powering and/or charging the apparatus 2, 2′, among other functions, as will be described in greater detail below. The motor housing 400 of apparatus 2 in FIG. 1 has a larger motor, and as such has a motor housing that is longer along a direction extending away from the user's face. The apparatus 2′ in FIG. 2 has a smaller motor than the apparatus of FIG. 1, and as such has a smaller motor housing 400′. The motor housing 400′ is at least shorter along a direction extending away from the user's face, as can be seen when comparing the motor housing 400′ in FIG. 2 to the motor housing 400 FIG. 1. When the apparatus 2′ of FIG. 2 is worn by the user, there may be significant benefits to having a smaller motor and smaller motor housing 400′ in relation to user comfort, ease of wearability, and the ability of the user to fall asleep and stay asleep for a longer period of time, among other benefits, as one of ordinary skill would understand. In one embodiment, the entire motor housing 400, 400′ can be wrapped in a silicone wrapping 7 or other protective material, such as rubber or compound of materials which will protect the motor housing 400, 400′. In one embodiment, the silicone wrapping 7 can be implemented by using two injection molded silicone pieces that are joined together by heat melting “rubber welding” around the seam between the two pieces.

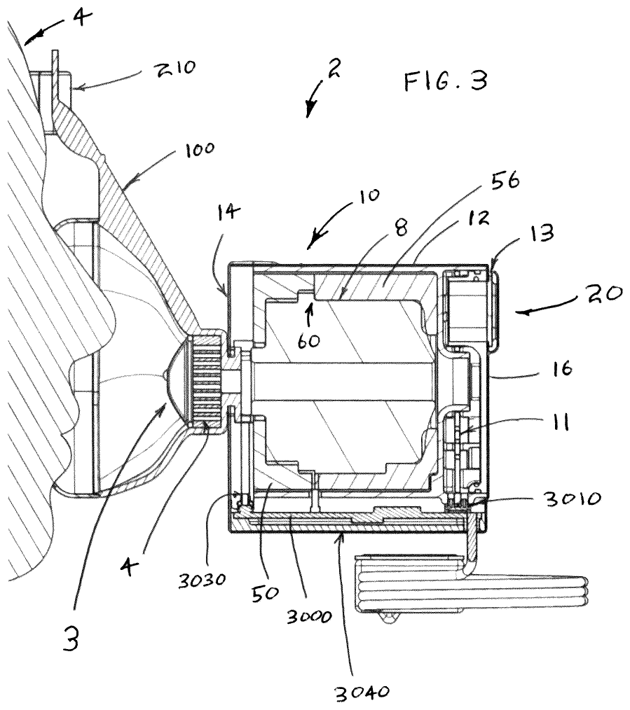

Referring to at least FIGS. 3, 4, and 5, one embodiment of the positive airflow pressure apparatus 2 is shown in cross-sectional and exploded views, respectively, showing the various internal parts of the apparatus 2. The apparatus can apply CPAP processes to assist the user with breathing, such as while a user sleeps, by at least generating a positive airflow pressure in the user's breathing air passage. The apparatus 2 includes the motor housing 10 (also shown in FIGS. 1 and 2 in the other embodiment of the apparatus 2 as elements 400, 400′). The motor housing 10, 400, 400′ can be made of lightweight yet solid and durable material, such as a polymer/plastic. The various portions of the motor housing 10, 400, 400′ be created using an injection molding process. Other creation processes, such as 3D printing, can be used as well.

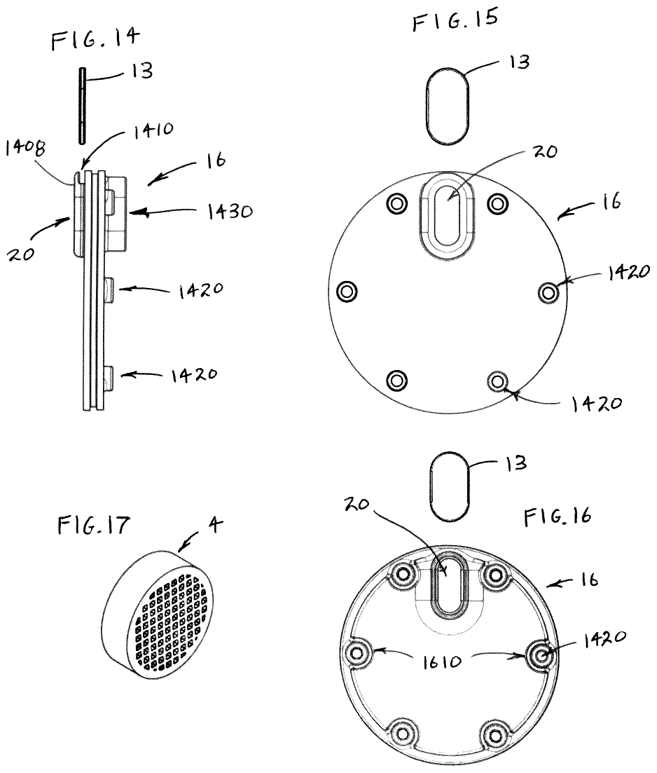

Referring additionally to FIGS. 14-16 and 25-28, the motor housing 10 has a main housing portion 12, an end cap 14, and an inlet plate 16. The inlet plate 16 can have a housing inlet 20 for allowing air to enter the motor housing 10 from outside the motor housing 10, such as when the user is inhaling. In one embodiment, the housing inlet 16 may also allow air to exit the motor housing 10, such as when the user is exhaling, although other embodiments have features which may restrict air from re-entering the motor housing 10 once the user inhales such air from the motor housing 10. The end cap 14 can have a housing outlet 22 for allowing air to exit the motor housing 10, such as when the user is inhaling. Likewise, in one embodiment, the housing outlet 22 may also allow air to enter the motor housing 10, such as when the user is exhaling, although other embodiments have features which may restrict air from re-entering the motor housing 10 through the housing outlet 22 once the user inhales such air from the motor housing 10 through the housing outlet 22.

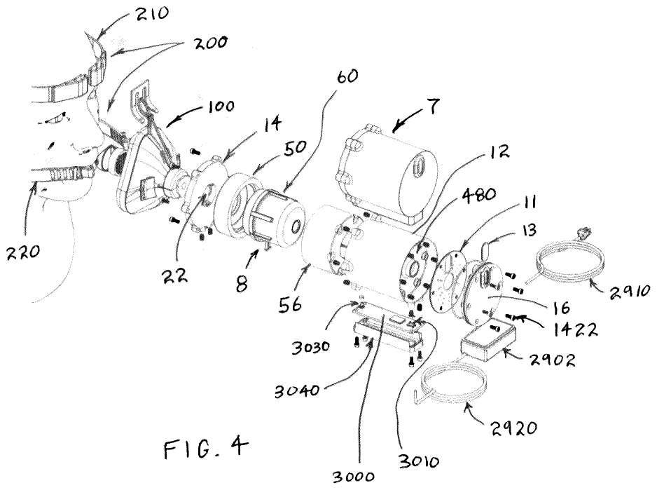

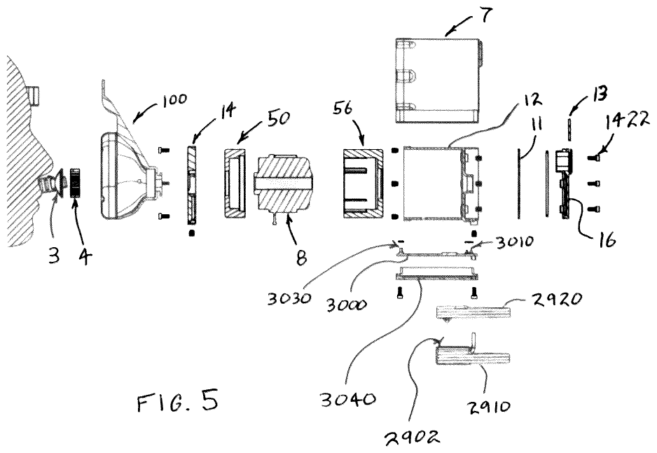

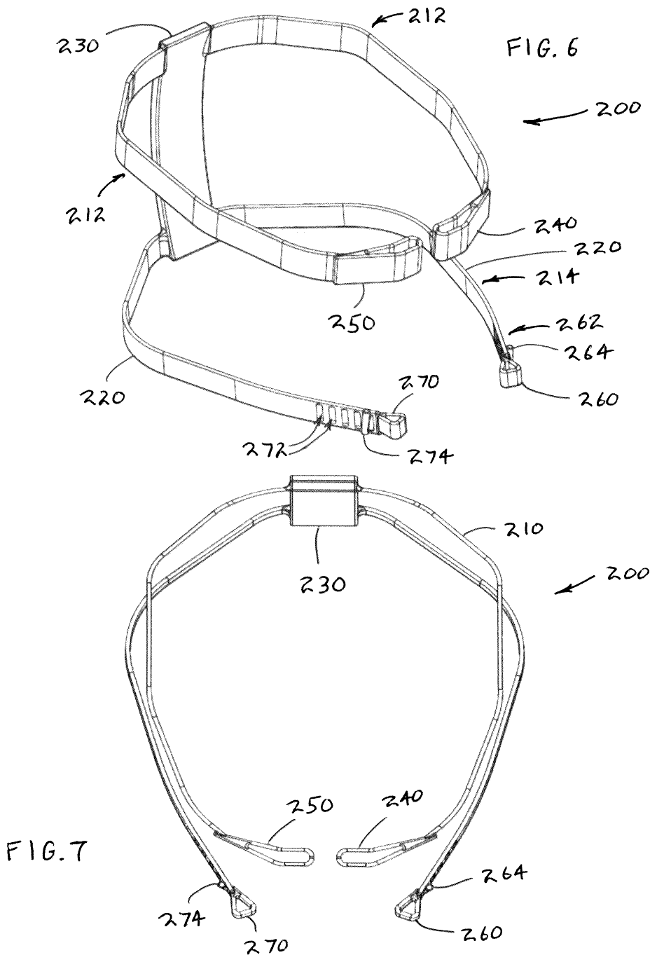

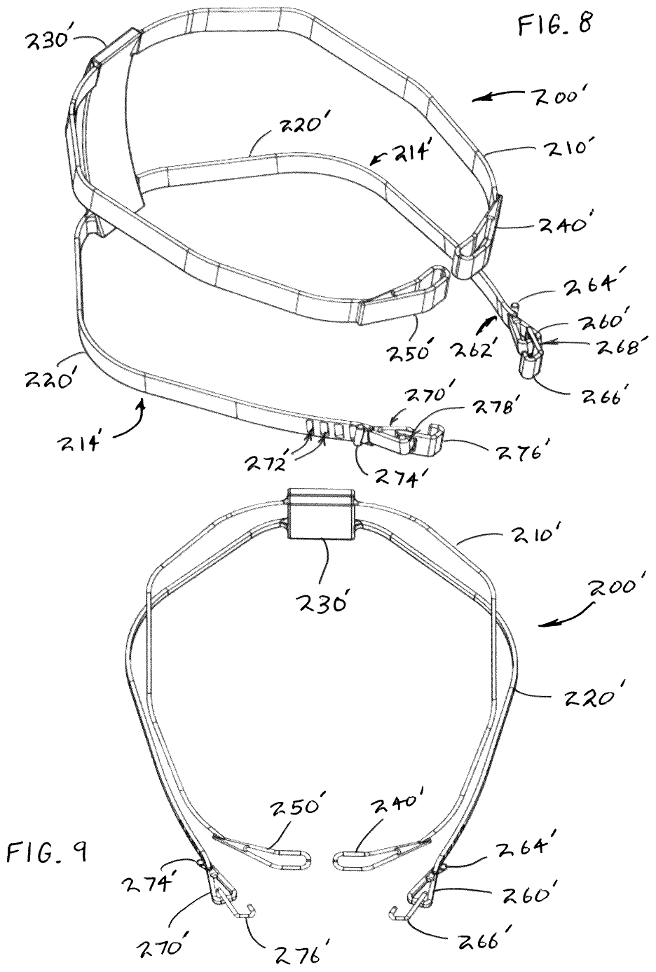

The inlet plate 16 can be removably connected to the main housing portion 12, and can be sealed thereto to assist in making the motor housing 10 substantially airtight. Specifically, the inlet plate 16 can be attached to the main housing portion 12 via screws 1422 and inlet plate screw holes 1420. The main housing portion 12 can have matching threaded screw receivers 1424, such as threaded inserts, for each inlet plate screw hole 1420 for receiving the screws 1422 in order to connect and secure the inlet plate 16 to the main housing portion 12, also securing the air restrictor plate 11 therebetween. Alternatively, the screw receivers 1424 can be self-tapping receivers, such as raw plastic undersized holes for screws to cut into. The inlet plate 16 can also have a removable air filter 13 for filtering air entering the motor housing 10 through the housing inlet 20. The air filter 13 can be inserted into an open end of a raised portion 1408 at the top of the raised portion 1408. The open end of the raised portion 1408 creates a filter slot 1410 for receiving the air filter 13 and for allowing removal of the air filter 13, for ease of replacement of the air filter 13. The inlet plate 16 can also have an inlet projection 1426 projecting from a rear side 1430 of the housing inlet 20. Air entering the housing inlet 20 exits on the opposite side of the inlet plate 16 through the inlet projection 1426, which will be further described below. A sealing gasket 520, shown in at least FIGS. 4 and 5, can be placed and secured in between the connection of the inlet plate 16 and the main housing portion 12, to at least further assist in making the motor housing 10 substantially airtight. In one embodiment, the sealing gasket 520 can be an O-ring.

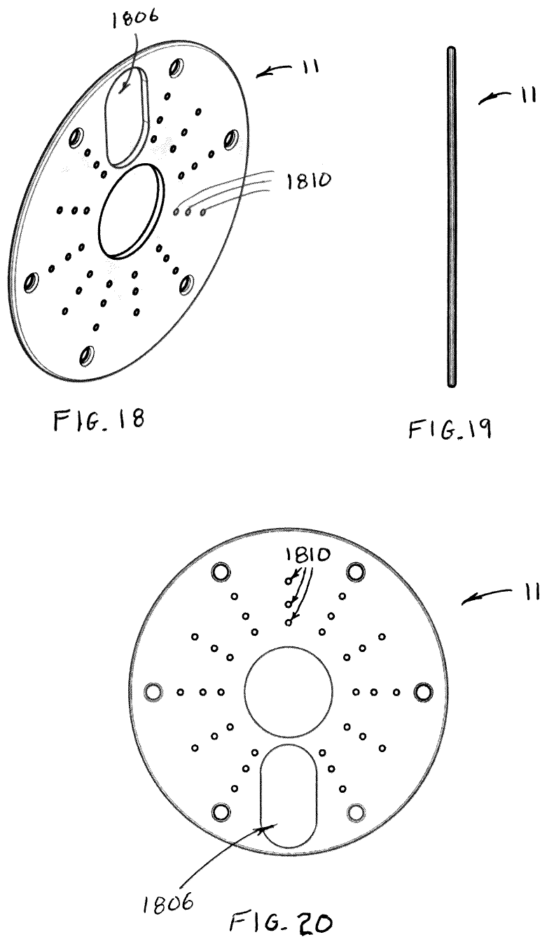

The main housing portion 12 has a vertical inner wall 480 from which the threaded screw receivers 1424 extend toward the inlet plate 16. The vertical inner wall 480 of the main housing portion 12 extends generally perpendicular to a tubular portion the main housing portion 12. The vertical inner wall 480 and the tubular portion define a housing front end chamber 500. Referring additionally to FIGS. 18-20, in a further embodiment, the apparatus 2, 2′ further has an air restrictor or barrier plate 11 secured in the housing front end chamber 500. The air restrictor plate 11 has a plurality of restrictor air holes 1810 for allowing air to pass from one side of the restrictor plate 11 to the opposite of the restrictor plate 11, and vice versa. The air restrictor plate 11 also has a plurality of screw holes 1820. Each screw hole 1820 receives a screw 1422 through a screw hole 1420 of the inlet plate 11, when the air restrictor plate 11 is inserted between the inlet plate 16 and the inner wall 480 of the main housing portion 12. In one embodiment, axial clamp of the air restrictor plate 11 can be achieved by having the bolt bores 1610 sandwich the air restrictor plate 11 on either side thereof.

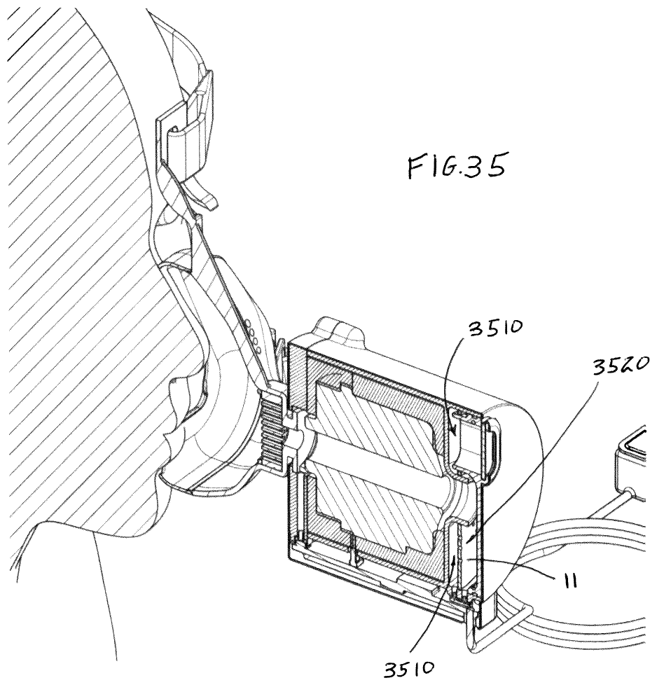

Referring additionally to FIG. 35, the air restrictor plate 11 separates the housing front end chamber 500 into a first front end chamber 3510 on a first side of the air restrictor plate 11 and a second front end chamber 3520 on a second side of the air restrictor plate 11. The plurality of restrictor air holes 1810 allow air to pass from the first front end chamber 3510 to the second front end chamber 3520, and vice versa. The air restrictor plate 11 also has an inlet projection receiver or inlet through hole 1806. When the inlet plate 11 is positioned within the front end chamber 500, the inlet projection receiver 1806 receives the inlet projection 1426 of the inlet plate 16, with the inlet projection 1426 positioned within the inlet projection receiver 1806. At least a portion of an outer side wall surface of the inlet projection 1426 fittingly engages with at least a portion of the an interior surface of the inlet projection receiver 1806.

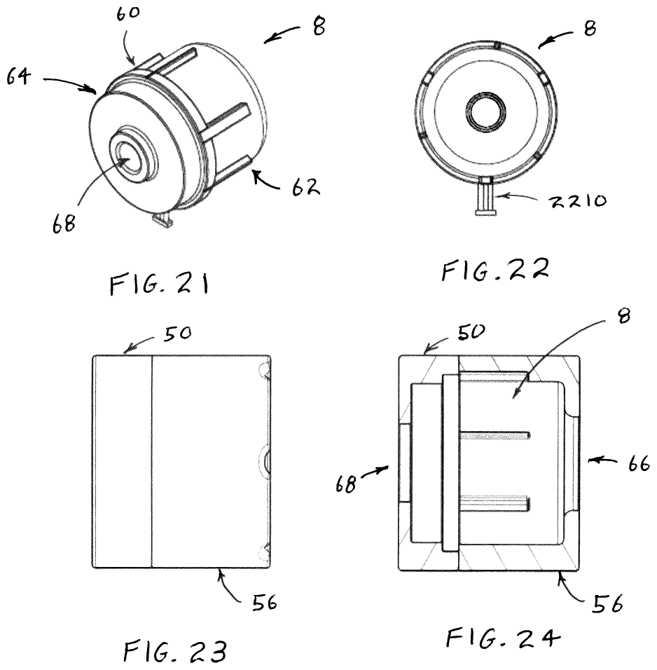

Referring additionally to FIGS. 21-24, the motor 8 is housed within the main motor housing 12. The motor 8 receives air from the housing inlet 20 and propels the air to the housing outlet 22. The motor 8 assists in creating a positive airflow pressure at the housing outlet 22. The motor 8 is preferably a variable speed motor, the speed of which is controlled by a controller, as described in more detail below. The motor 8 can take various forms and can be oriented within the motor housing 10 and main housing portion 12 in various different ways. Specifically, the motor 8 can be an axial flux blower motor, a radial flux blower motor, an axial blower motor, or a radial blower motor. The orientation and placement of the each of these types of motors within the motor housing 10/main housing portion 12 will depend on various factors, including at least the preferred direction of air flow, noise generation by each motor in each orientation, air flow speed, rate of change of the speed of the motor, among various other factors a designer may use or choose to consider. In one preferred embodiment, the motor 8 is an AIRSENSE 10 axial blower motor, made by HAMEISEN of China, ASIN B0DN6V9N5B, with dimensions of 3.7×4.46×3.31 inches, weighing 8.47 ounces.

The main housing portion 12 also has a motor chamber 540 located on the opposite side of the inner wall 480 from the housing front end chamber 500. The apparatus 2, 2′ also has a first sealing gasket 50 and second sealing gasket 56 within the motor chamber 540. In one embodiment, the sealing gaskets 50, 56 can be made of food grade, medical grade, and/or non-toxic silicone. The first sealing gasket 50 circumferentially surrounds an outlet portion of the motor 8 and the second sealing gasket 56 circumferentially surrounds an inlet portion of the motor 8 within the motor chamber 540. In one embodiment, the motor chamber 540 receives the motor 8 encased within the first sealing gasket 50 and the second sealing gasket 56. The first sealing gasket 50 encases the outer surfaces of a first end of the motor 8 toward an outtake 68 of the motor 8, and the second sealing gasket 56 encases the outer surfaces of a second end of the motor 8 toward the intake 66 of the motor 8. When the first end of the motor 8 is inserted into the first sealing gasket 50 and the second end of the motor 8 is inserted into the second sealing gasket 56, the inner side surfaces of each of the first and second sealing gaskets 50, 56 can meet, engage, or abut one another, or just be adjacent one another, at a central portion 60 of the motor 8. This engagement assists in reducing any motor noise outside of the motor housing 10 and seals the motor at the inlet and outlet interfaces, allowing for air to flow through the motor housing 10 only through the intake 66 and outtake 68 of the motor 8. While some motors are fairly quiet, most motors make at least some noise, whereby the use of such motors in a substantially dead silence is detectable by the human ear. As such, the use of the first and second sealing gaskets 50, 56 is preferable to reduce such noise. In many of the embodiments of the present invention, noise reduction can be even more significant, at least given that the motor 8 is closer to the user's ears when the apparatus 2, 2′, including the motor 8, is worn by the user. It should be understood that the shape and design of the first and second sealing gaskets 50, 56 will at least depend on the type and shape or the motor 8, as well as the placement and orientation of the motor 8 within the motor housing 10. With the motor 8 encased within the first and second sealing gaskets 50, 56, the first and second sealing gaskets 50, 56 are inserted into the motor chamber 540 of the motor housing 10, as shown at least FIG. 3. When the motor 8 also has a control connection 2210 for operably connecting the motor 8 to a controller or onboard control unit, as described below. Through the control connection 2210, the controller controls the operation of the motor 8, such as controlling the speed of the motor 8 when the motor 8 is a variable speed motor. The control connection 2210 can be one or more low voltage wires or leads or can be in the form of pneumatic connectors when the control is performed by pneumatic control methods. Other forms of control can be used as well, such as wireless control.

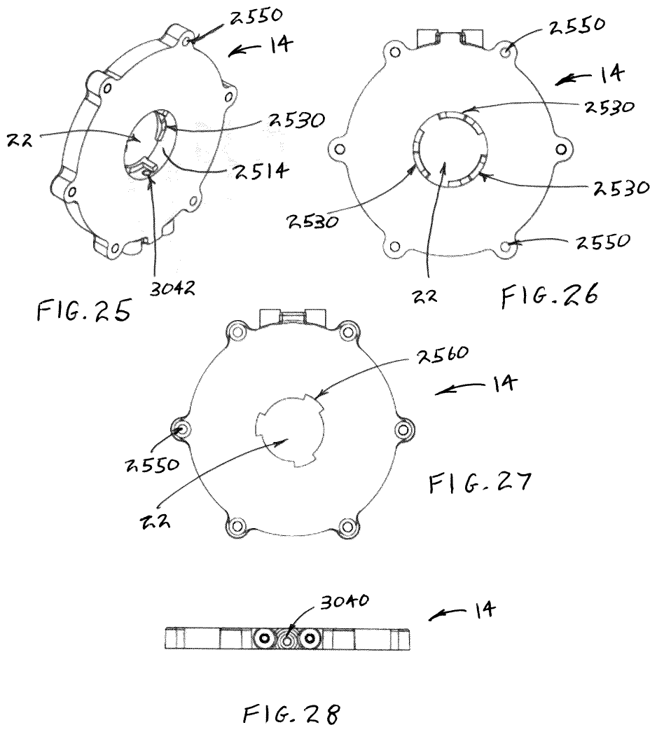

Referring again to FIGS. 25-28, the motor housing 10 also has an end cap 14 for enclosing and capping the motor 8, as encased within the first and second sealing gaskets 50, 56 within the motor chamber 540 of the main housing portion 12. Similar to the inlet plate 16, the end cap 14 has a plurality of end cap screw holes 2550 for removably securing the end cap 14 to the main housing portion 12 with screws shown in both FIGS. 4 and 5. The housing outlet 22 is provided in a central portion of the end cap 14. On one embodiment, the end cap 14 has an interior side wall 2514 forming the housing outlet 22. The interior side wall 2514 has a plurality of flanges 2530 extending inward and along the surface of the interior side wall 2514. The plurality of flanges 2530 form cap notches 2560 on the exterior surface of the end cap 14 and motor housing 10 proximate the housing outlet 22. The cap notches 2560 and plurality flanges 2530 function to receive and connect the face mask 100 to the motor housing 10, as will be described in more detail below. The end cap 14 also has an end cap air pressure passage vertically formed within the end cap 14 for allow air to flow from the housing outlet 22 through an end cap air pressure passage inlet 3042 to an end cap air pressure passage exit 3040, for assisting in detecting and determining air pressure proximate the housing outlet 22, as will be described in greater detail below.

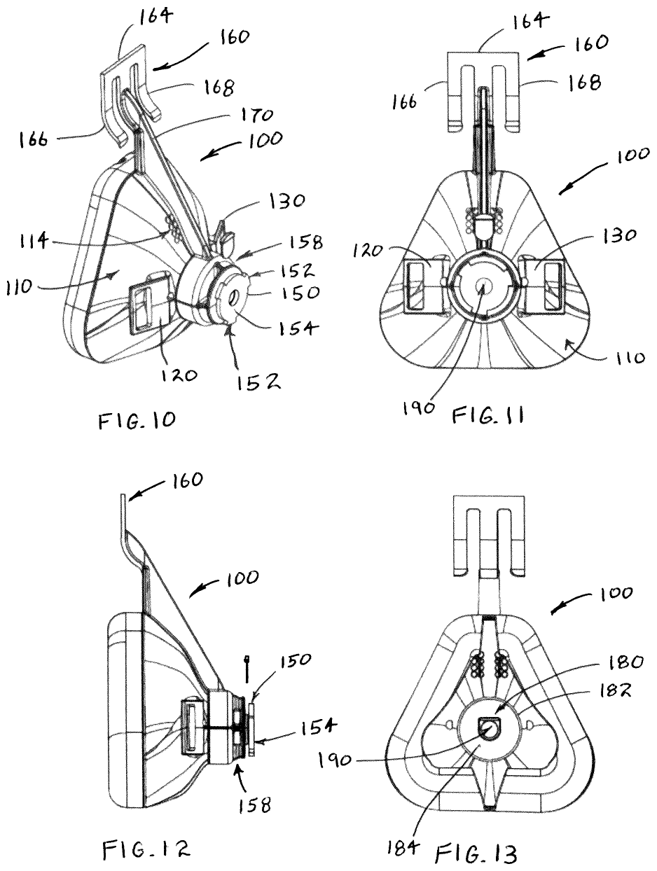

Referring additionally to FIGS. 10-13, the apparatus also includes a face mask 100 for attachment to the user's face and for engaging with one or both of the user's air breathing passages. In one embodiment, the face mask 100 has a main mask portion 110 for covering the nose and mouth air passages of a user, as shown in at least FIGS. 1 and 2. Like other elements of the apparatus 2, 2′, the face mask 100 can be formed using an injection molding process and made of a material that firm enough to support the motor housing 10, 400, 400′ when connected to the face mask 100. The face mask 100 can have a connection receiver 1100 for receiving and attaching a face mask connector 200 to the face mask 100, for physically attaching the face mask 100 and the motor housing 10, 400, 400′ to the user. In one embodiment, the connection receiver 1100 includes a first connection receiver 120 located and attached to a left front side of the main mask portion 110. The connection receiver 1100 also includes a second connection receiver 130 located and attached to a right front side of the main mask portion 110. The connection receiver 1100 further includes a third connection receiver 160 located and attached to a front top portion of the main mask portion 110 by way of a support 170. In one embodiment, the support 170 is attached to the main mask portion 110 from a central area of the front side of the main mask portion 110 all the way to a top of the front side of the main mask portion 110. The support 110 can take the form of vertically oriented plane or plastic/polymer sheet extending away from the main mask portion 110 from the front top portion of the main mask portion 110. The bottom of the support 110 can also be attached to a top portion of an exterior surface of an air treatment chamber side wall 182, as shown in at least FIG. 10.

In one embodiment, the third connection receiver 160 can have a T-joint 164 which is attached to and extends from the top of the support 110. The third connection receiver 160 can also have a first arm 166 which is attached to and extends downwardly from the left side of the T-joint 164. The third connection receiver 160 can also have a second arm 168 which is attached to and extends downwardly from the right side of the T-joint 164. Each of the first and second arms 166, 168 can be configured or formed to include a frontward bend toward the bottom of each of the first and second arms 166, 168, for preventing the face mask connector 200 from disengaging from the first and second arms 166, 168, and for better securing the face mask connector 200 to the face mask 100 and the face mask 100 to the user's head.

As will be better understood with reference to the below description of the face mask connector 200 and FIGS. 6-9, the first connection receiver 120 can receive and allow for removable attachment to a left portion of a bottom connector 220 of the face mask connector 200 to the face mask 100. Likewise, the second connection receiver 130 can receive and allow for removable attachment to a right portion of the bottom connector 220 of the face mask connector 200 to the face mask 100. The third connection receiver 160 can receive and allow for removable attachment of left and right portion of a top connector 210 of the face mask connector 200 to the face mask 100. Specifically, the first arm 166 of the face mask 100 receives and allows for removable attachment of the left portion of the top connector 210 of the face mask connector 200 to the face mask 100. The second arm 168 of the face mask 100 receives and allows for removable attachment of the right portion of the top connector 210 of the face mask connector 200 to the face mask 100.

In one embodiment of the apparatus 2, 2′, the main mask portion 110 of the face mask 100 further has air holes 114 for allowing at least some of the user's exhaled air to exit the face mask 100 through the air holes 114 when the user exhales. In one embodiment, the face mask 100 can further include an air restrictor 158 attached to a front portion of the main mask portion 110 of the face mask 100 proximate a bore 190 of the face mask 100 for allowing air to enter the face mask 100 from the motor housing 10, 400, 400′, and for preventing air from flowing back into the motor housing 10 from the face mask 100. The air restrictor 158 can be in the form of a one-way air restrictor or restrictor check, or other type of air restrictor 158. The selection of whether to use an air restrictor 158 and the type of air restrictor 158 depends on various factors, including the selected method of operation and/or algorithms used for the operation of the apparatus 2, 2′ as will be described further below. In one embodiment, the air restrictor 158 can be a one-way flow valve or a silicon flap that folds up to open the air passage between the face mask 100 and the motor housing 10, 400, 400′ when the user inhales or the user's breathing is neutral, but closes when the user exhales.

The face mask 100 can also have an air treatment chamber side wall 182 extending from a front portion of the main mask portion 110 of the face mask 100. The face mask 100 can further have an air treatment chamber front wall 154 extending from the air treatment chamber side wall 182 to create an air treatment chamber 180 for receiving an air treatment device 4 (shown in FIG. 17). The air treatment device 4 is preferably a dehumidification device or a humidification device, for reducing or increasing, respectively, the humidity of the air entering the user's air passage. The air treatment device 4 can also include air filtering aspects or can be an air filter with de-humidification or humidification aspects as well. The air treatment device 4 can also be of an antibacterial material or have antibacterial coatings added to the air treatment device 4 during or after manufacture for ensuring only purified air can re-enter the motor housing 10, 400, 400′. A clip 3 or other securing device can be used to secure the air treatment device 4 within the air treatment chamber 180.

In one embodiment of the apparatus of the present invention, the face mask 100 further has a housing connector 150 attached to a front portion of the main mask portion 110 of the face mask 100. In one embodiment, the housing connector 150 can be connected to and extend from the opposite side of the air treatment front wall 184 and air treatment chamber 180 and/or from the air restrictor 158. The housing connector 150 can have a flat front wall 154 and a plurality of tabs 152 extending from a perimeter of the flat front wall 154, for removably connecting the face mask 100 to the motor housing 10, 400, 400′. Specifically, in one embodiment, in order to removably connect the face mask 100 to the motor housing 10, 400, 400′, each of the plurality of tabs 152 can be inserted into one of the plurality of the cap notches 2560 of the end cap 14. Once the plurality of tabs 152 are inserted into one of the plurality of the cap notches 2560, the motor housing 10, 400, 400′ and/or the face mask 100 can be rotated thereby causing the plurality of tabs 152 to engage with the plurality of flanges 2530. The plurality of flanges 2530, which extend from the interior side wall 2514 of the end cap 14, receive the respective tabs 152 of the face mask 100 for allowing the secure, yet removable, attachment of the motor housing 10, 400, 400′ to the face mask 100.

A bore 190 is formed through a central portion the housing connector 150 for allowing air to flow between the housing connector 150 and face mask 100, and the motor housing 10 through the housing outlet 22. The face mask 100 is removably attachable to the motor housing 10, 400, 400′ proximate the housing outlet 22, with the face mask 100 receiving the propelled air from the motor 8 through the housing outlet 22 and the bore 190 of the face mask 100. As such, at least the motor 8 and the face mask 100, as attached to one another as described herein above in one embodiment, operate together to generate the positive airflow pressure in the user's breathing air passage. The apparatus 2, 2′ can be used without separating the motor 8 and motor housing 10, 400, 400′ (and control unit (described below)) from the face mask 100. In one embodiment, the face mask 100, motor 8 inside the motor housing 10, 400, 400′, and control unit (that is a part of the motor housing 10, 400, 400′) are all worn by the user as an integral apparatus. However, at least the face mask 100 and the motor housing 10, 400, 400′ (with the motor 8 therein) can be removably attached and connected to one another as described above herein, to at least allow for ease of cleaning and/or replacement of the face mask 100, or certain aspects thereof.

The apparatus 2, 2′ further has a face mask connector 200, 200′ for physically and removably attaching the face mask 100 and the motor housing 10, 400, 400′ to the user, and for allowing the user to wear the apparatus 2, 2′. FIGS. 6 and 7 show one embodiment of the face mask connector 200 and FIGS. 8 and 9 show another embodiment of the mask connector 200′. In one form of these embodiments, the face mask connector 200, 200′ has a top connector 210, 210′ with a top elongated portion 212, 212′, a left closed end loop 250, 250′ toward a left end of the top elongated portion 212, 212′, and a right closed end loop 240, 240′ toward a right end of the top elongated portion 212, 212′. Alternatively, the left closed end loop 250, 250′ and the right closed end loop 240, 240′ (and other ends of the face mask connector 200, 200′) are not “closed,” are of a flexible material, and can each have removable attachment means, such as Velcro, for performing the same/similar functions as described. As was described above, the left closed end loop 250, 250′ and the right closed end loop 240, 240′ can be removably attached to the first arm 166 and the second arm 168 of the third connection receiver 160 of the face mask 100 respectively for removeable attachment of the face mask 100 and motor housing 10, 400′ 400′ to the user. As such, the left closed end loop 250, 250′ and the right closed end loop 240, 240′ are for securing and connecting the top connector 210, 210′ to the third connection receiver 160 of the face mask 100.

The face mask connector 200, 200′ further has a bottom connector 220, 220′ with a bottom elongated portion 214, 214′. The face mask connector 200, 200′ also has a left open end loop 270, 270′ toward a left end of the bottom elongated portion 214, 214′, and a right open end loop 260, 260′ toward a right end of the bottom elongated portion 214, 214′. The face mask connector 200, 200′ also has a joiner 230 for connecting the top elongated portion 212, 212′ of the top connector 210, 210′ to the bottom elongated portion 214, 214′ of the bottom connector 220, 220′, for maintaining generally consistent distance between the top elongated portion 212, 212′ and the bottom elongated portion 214, 214′. The joiner 230 can be fixed to and/or formed integral with the top elongated portion 212, 212′ toward the top of the joiner 230. Alternatively, the joiner 230 can have a top slot toward the top of the joiner 230 for allowing the top elongated portion 212, 212′ to be threaded therethrough and to be in slidable engagement with the joiner 230, for allowing the user to adjust the position of the joiner 230 relative to the top elongated portion 212, 212′. The joiner 230 can also be fixed to and/or formed integral with the bottom elongated portion 214, 214′ toward the bottom of the joiner 230. Alternatively, the joiner 230 can have a bottom slot toward the bottom of the joiner 230 for allowing the bottom elongated portion 214, 214′ to be threaded therethrough and to be in slidable engagement with the joiner 230, for allowing the user to adjust the position of the joiner 230 relative to the bottom elongated portion 214, 214′. Alternatively, as mentioned, Velcro can instead be used to resize/properly size the face mask connector 200, 200′ to the face mask 100.

In one embodiment, the bottom connector 220, 220′ can have a plurality of left through holes 272, 272′ toward the left end of the bottom elongated portion 214, 214′, and a plurality of right through holes 262, 262′ toward the right end of the bottom elongated portion 214, 214′. The plurality of left through holes 272, 272′ and right through holes 262, 262′ can be used to engage and connect with the left open end loop 270, 270′ and right open end loop 260, 260′, respectively. Specifically, the left open end loop 270, 270′ can have a left end dowel 274, 274′ and the right open end loop 260, 260′ can have a right end dowel 264, 264′. The left end dowel 274, 274′ can be inserted into at least one of the plurality of left through holes 272, 272′ for securing and connecting the left end dowel 274, 274′ and respective left open end loop 270, 270′ to the first connection receiver 120 of the face mask 100, after looping the left end dowel 274, 274′ through the first connection receiver 120. Likewise, the right end dowel 264, 264′ can be inserted into at least one of the plurality of right through holes 262, 262′ for securing and connecting the right end dowel 264, 264′ and respective right open end loop 260, 260′ to the second connection receiver 130 of the face mask 100, after looping the right end dowel 264, 264′ through the first connection receiver 130. The user can adjust the tightness of the face mask 100 to the user's face by selecting a specific right through hole 262 and left through hole 272 to insert the respective right end dowel 264 and left end dowel 274, for securing the face mask 100 and moto housing 10 to the user's face.

In a further embodiment shown in FIGS. 8 and 9, the bottom connector 220′ can have a left hook 276′ with a left opening 278′ and a right hook 266′ with a right opening 268′. The left open end loop 270′ can be inserted within the left opening 278′ and the right open end loop 260′ can be inserted in the left opening 268′. The left hook 276′ can be inserted in and attached to the first connection receiver 120 for securing and connecting the left open end loop 270′ and bottom connector 220′ to the first connection receiver 120 of the face mask 100. Likewise, the right hook 266′ can be inserted in and attached to the second connection receiver 130 for securing and connecting the right open end loop 260′ and bottom connector 220′ to the second connection receiver 130 of the face mask 100.

Referring again to FIGS. 3-5 and 35, and additionally to FIGS. 29-31, the positive airflow pressure apparatus 2, 2′ can have a power and control apparatus 2900 for powering and controlling the speed and direction of the motor 8. The power/control apparatus 2900 can have an external controller/charger 600, 2902 connected to a power source via a power cord 2910. The power cord 2910 will typically be connected to a 110 V or 220 V power connection and the external controller/charger can include an AC to DC converter to power various aspects of the apparatus using DC power, as one of skill in the art would understand. The power/control apparatus 2900 also has a power/control cord 2920 for removably connecting the external controller/charger 2902 to an onboard printed circuit board (PCB) or control board 3000 via a power/control terminal 3050. The onboard control board 3000 is a part of the motor housing 10, 400, 400′ and is mounted and secured to the bottom of the motor housing 10, 400, 400′. A control board housing 3040 covers and protects the onboard control board 3000, and along with the onboard control board 3000, the control board housing 3040 is mounted to the bottom of the motor housing 10, 400, 400′ using screws or other securing means, as shown in FIG. 4.

The control board 3000 can include an onboard controller 3020, a differential pressure sensor 3010, and an air pressure sensor 3030. In one embodiment, the control board 3000 does not include the onboard controller 3020, with the functions of the onboard controller 3020 taking place with the external controller/charger 2902, and with leads extending from control board 3000, including from the differential pressure sensor 3010 and the air pressure sensor 3030, to the external controller/charger 2902. Returning to the embodiment with the control board including the onboard controller 3020, the onboard controller 3020 can have computer memory for storing software and/or firmware, including algorithms, for carrying out and performing known and other positive airflow pressure control operations and algorithms. The software and/or firmware can be programmable and can allow for customizations for each user's attributes and conditions, each of which may require that, and/or allow for, certain custom settings to be used for each user. The mode of operation, such as a specific positive airflow pressure control operation and/or algorithms can be selected and run, and/or custom settings can be input and modified for each user's attributes, using the external controller/charger through the control display 2904, which can take the form of a touch screen input/output display. Alternatively or additionally, the mode of operation, such as a specific positive airflow pressure control operation and/or algorithms can be selected and run, and/or custom settings can be input and modified for each user's attributes, using a remote mobile App or computer software application running on a standard computer, which can communicate with the external controller/charger 2902 through a WiFi, BLUTOOTH, or other wireless or wired connection between the external controller/charger 2902 and the Internet. As such, the external controller/charger 2902 can include the necessary hardware and firmware/software to carry out such functions, as described further below.

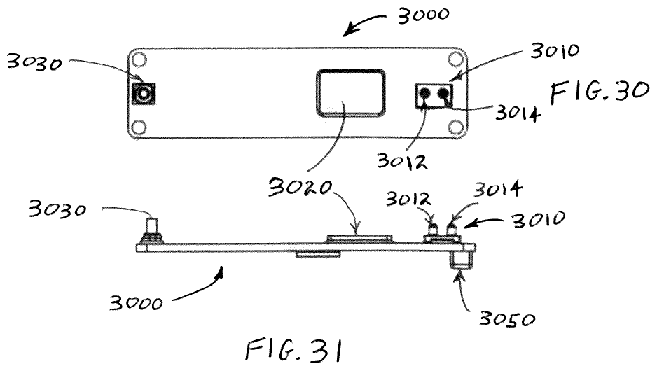

In one embodiment, the differential pressure sensor 3010 is built into the control board 3000 toward one end so that when the control board 3000 is secured in the motor housing 10, the differential pressure sensor 3010 is positioned between the first front end chamber 3510 and the second front end chamber 3520 of the motor housing 10 for sensing a pressure differential between the first front end chamber 3510 and the second front end chamber 3520, as air flows between the first and second front end chambers 3510, 3520 through the restrictor air holes 1810 of the air restrictor plate 11. In one embodiment, the differential pressure sensor 3010 has a first differential input 3012 and a second differential input 3014, as shown in at least FIGS. 30-31, which when the control board 3000 is secured in the motor housing 10, the first differential input 3012 is located within the first front end chamber 3510, and a second differential input 3014 is located within the second front end chamber 3520. The differential pressure sensor 3010 continuously detects the pressure difference between the first front end chamber 3510 and the second front end chamber 3520 and sends a signal representing that difference to the onboard controller 3020, which in turn, determines the pressure difference. The pressure difference is then used as a part of the control method and/or algorithm being used for controlling the operation of the motor 8, including at least the motor direction and motor speed on a continuous basis.

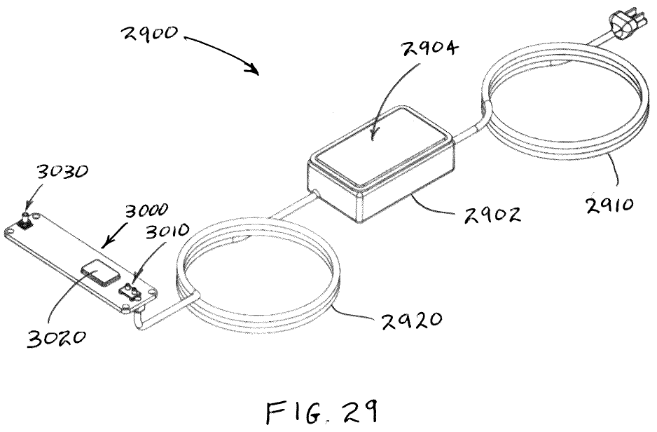

In one embodiment, the air pressure sensor 3030 is also built into the control board 3000 toward the opposite end from the differential pressure sensor 3030 so that when the control board 3000 is secured in the motor housing 10, the air pressure sensor 3030 is positioned to be inserted within and engage with the end cap air pressure passage exit 3040 shown in FIG. 28, which detecting the air pressure proximate the housing outlet 22 via end cap air pressure passage entrance 3042. The air pressure sensor 3030 continuously detects this air pressure and sends a signal representing that air pressure to the onboard controller 3020, which in turn, determines the air pressure. The air pressure is then used as a part of the control method and/or algorithm being used for controlling the operation of the motor 8, including at least the motor direction and motor speed on a continuous basis. Thus, the onboard controller 3020 can continuously determine and control the speed of the variable speed motor form of the motor 8 in response to the sensed air pressure and the sensed pressure differential, for generating the positive airflow pressure in the user's air passage.

In one embodiment, the apparatus 2, 2′ can have an rechargeable battery connected to and powering the control board 3000/motor driver 3850/motor 8, which can either reside in the external controller/charger unit 2902 or on the control board 3000. The rechargeable battery can be built into the control board 3000 or be located/positioned elsewhere within the apparatus 2, 2′ such as inside the external controller/charger unit 2902 with leads running from the rechargeable battery to the control board 3000/motor driver 3850/motor 8. A charger, which can be built into the external controller/charger 2902, can be removably attached/connected to the rechargeable battery, to recharge the rechargeable battery when the battery charge becomes low. In another embodiment, the apparatus has a wired power module, such as the external controller/charger 2902, connected to and powering the control board 3000, onboard controller 3020, and motor driver 3850/motor 8. In various embodiments, the motor driver 3850 can be located on the control board 3000 or within the external controller/charger 2902. The battery powered apparatus can also function while being charged in a pass-through charging configuration.

The operational and functional features of the onboard controller 3020 can alternatively be provided in a non-onboard controller, such as the external controller/charger 2902 in addition to and/or instead of the onboard controller 3020, with the onboard controller 3020 having limited functionality relative to the motor functions and relative to receiving signals received from the differential pressure sensor 3010 and the air pressure sensor 3030.

Referring additionally to FIGS. 36-38, in operation, when the user inhales, air enters the housing inlet 20. The air travels through the air filter 13 and then through the inlet projection 1426. The air then enters the first front end chamber 3510 on one side of the air restrictor 11, and then through the restrictor air holes 1810 and into the second front end chamber 3520 on the opposite side of the air restrictor 11. The air then moves into the intake 66 of the motor 8 through the motor 8, which accelerates the air and creates air pressure, and then exits the motor 8 through the outtake 68 of the motor 8. The air then moves through the housing outlet 22 and into the bore 190 of the face mask 100, and then through the antibacterial/humidification/dehumidification filter 4 located in the air treatment chamber 180, after which the air moves into one or more of the user's breathing passages. In one embodiment, when the user exhales, the referred to air restrictor 158 stops the air from flowing back into the motor housing 10, 400, 400′ and forces the air out of the air holes 114 in the face mask 100. In another embodiment, when the user exhales, some of the cleaned, filtered air can move back into the motor housing 10, 400, 400′, although, depending on how the apparatus 2, 2′ is programed to operate, the motor 8 can be controlled to maintain a continuous positive air pressure in the direction the face mask 100, still forcing at least some of the air to exit the face mask 200 through air holes 114. In one embodiment, airflow during inhalation and exhalation is read differently at the differential pressure sensor 3010 mounted across the airflow restrictor 11. In particular, the data from the differential pressure sensor 3010 when processed allows for the detection of inhalation versus exhalation condition. The air pressure sensor 3030 monitors the pressure inside the face mask 100 and data from which can also be used in the algorithms used to detect inhalation versus exhalation condition.

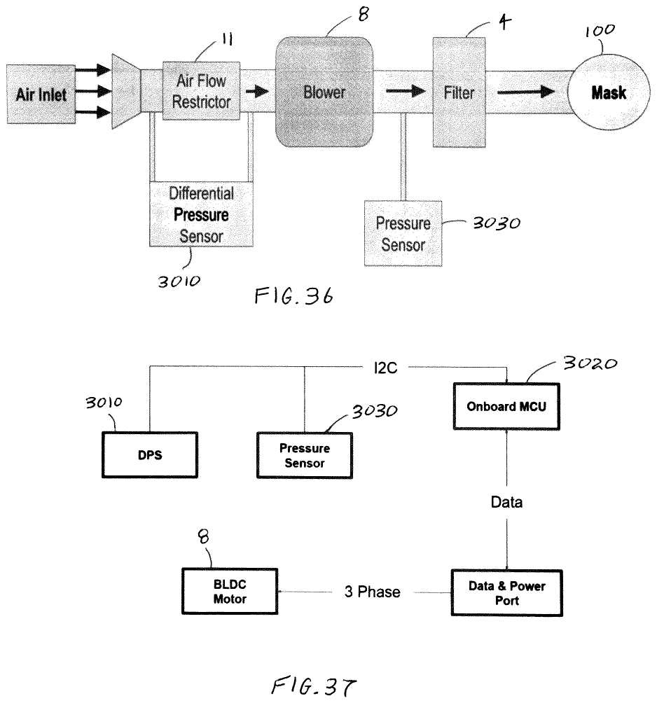

Referring to FIG. 37, in one embodiment, the air pressure sensor 3030 and differential pressure sensor 3010 provide real time feed back to the onboard controller 3020, allowing for the maintenance of a desired airway pressure dynamically in response to the user's breathing. The control board 3000 can include voltage regulators to step down a 24V supply to 5V for internal components. Additionally, a dedicated communication port allows for software/firmware upgrades and facilitates data exchange with the external controller/charger 2902.

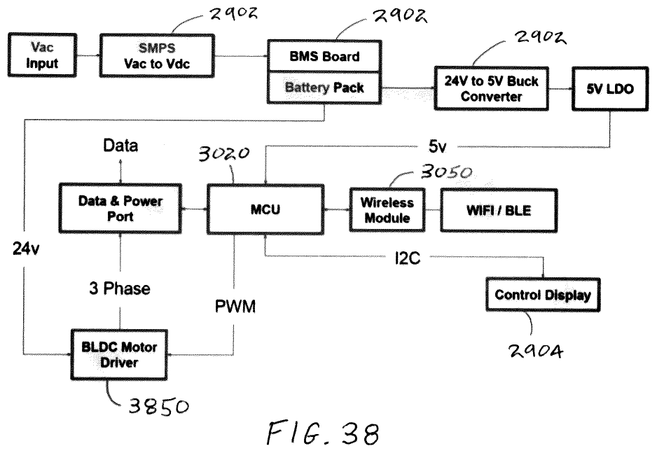

Referring to FIG. 38, in one embodiment, the external controller/charger 2902 can serve as a user interface through control display 2904 and as a control center for the apparatus 2, 2′. The external controller/charger 2902 can include an AC to DC converter to generate stable 24V output. A separate microcontroller manages display functions, system settings, and communication tasks. The external controller/charger 2902 can include a WiFi module to support over the air (OTA) updates along with 24V to 5V regulators to power internal electronics. The external controller/charger 2902 can handle non-therapy operations, such as user interaction, parameter adjustments, data logging, and wireless connectivity. The external controller/charger 2902 can function as a bridge between the user and the therapy operations, sending updated parameters and commands to the control board 3000 through the digital communications link. A battery pack, such as a rechargeable lithium ion or other rechargeable battery pack, can be included within the external controller/charger 2902 and/or within the control board 3000, or elsewhere within the motor housing 10, 400, 400′.

As mentioned, the apparatus 2, 2′ and onboard controller 3020 and/or external controller/charger 2902, can be programmed to operate using different operational methods. In one example of a method of operation of the apparatus 2, 2′ the onboard controller 3020 and/or external controller/charger 2902 can carry out a fixed-pressure CPAP method, in which the onboard controller 3020 and/or external controller/charger 2902 controls the motor driver 3850, which in turn drives/controls the speed and/or the direction of the motor 8. A fixed-pressure CPAP method delivers a single, prescribed air pressure level determined by a sleep specialist during a CPAP titration study. The apparatus draws in room air, filters it, and pressurizes it to the set level before delivering it through the face mask 100.

In another example of a method of operation of the apparatus 2, 2′ the onboard controller 3020 and/or external controller/charger 2902 can carry out an auto-adjusting PAP (APAP) or Auto-CPAP method. An APAP method automatically adjusts the pressure within a prescribed range based on the user's breathing patterns and airway resistance. This allows the machine to provide the optimal pressure needed at any given time during the night.

In a further example of a method of operation of the apparatus 2, 2′ the onboard controller 3020 and/or external controller/charger 2902 can carry out a Bilevel PAP (BiPAP) method. A BiPAP method uses two pressure levels: a higher pressure during inhalation (IPAP) and a lower pressure during exhalation (EPAP). A higher pressure during inhalation (IPAP) and a lower pressure during exhalation (EPAP), sometimes referred to as “pressure relief” or EPR (expiratory pressure relief), can also be implemented in other modes, such as CPAP mode (i.e., “CPAP with EPR”).

When the apparatus 2, 2′ is programmed to utilize an auto-adjusting CPAP algorithm, the apparatus 2, 2′ can employ sophisticated algorithms to monitor breathing and adjust pressure in real-time. These algorithms typically analyze several parameters to detect and respond to sleep-disordered breathing events. When the apparatus 2, 2′ is programmed to utilize a breathing pattern analysis algorithm, the apparatus 2, 2′ monitor breathing on a breath-by-breath basis, evaluating factors such as airflow, changes in flow patterns, and the presence of flow limitation. Further, when the apparatus 2, 2′ is programmed to utilize an event detection algorithm, the apparatus 2, 2′ can detect events such as apnea, hypopnea, snoring, flow limitations, respiratory effort related arousal (RERA), and/or effects related to COPD. When the apparatus 2, 2′ is programmed to utilize a pressure adjustment analysis algorithm, the apparatus 2, 2′ can detect an obstructive event and adjust the pressure upwards to keep the airway open. Once the breathing stabilizes, the apparatus 2, 2′ can be programmed to gradually decrease the pressure. When the apparatus 2, 2′ is programmed to utilize a response differentiation algorithm, the apparatus 2, 2′ can differentiate between obstructive events and other factors like mask leaks or central apneas, tailoring the response accordingly. For instance, if a central apnea is detected, the apparatus 2, 2′ can be programmed to not increase pressure. The apparatus 2, 2′ and onboard controller 3020 and/or external controller/charger 2902 can also be programmed operate using combinations of two or more of the above methods of operation and algorithms as well.

As mentioned, the various parts of the present invention can be injection molded and/or created using 3D printing techniques, using materials such as polymers, plastics, silicones or other rubbers or other suitable materials. However, those skilled in the art will appreciate that the various parts can be constructed from a variety of other suitable materials and with a variety of other suitable manufacturing techniques.

It will be understood that as used herein, the terms “controller,” “computer,” and “server” are synonymous, and refer to a microprocessor operating computer software that is configured to perform the software tasks described herein. The apparatus 2,2′ as described previously herein, is electronically controlled, and is equipped for that purpose with electronic circuitry including the control board 3000, the onboard controller 3020, and the external control/charger 2902. The external control/charger 2902 is responsible for generating the control display or user interface 2904, processing commands received from the user interface 2904, displaying information to the user and communicating with onboard controller 3020 and remote App/computer application.

Generally, in terms of hardware architecture the controllers include a processor, memory, and one or more input and/or output (I/O) devices (or peripherals), such as the user interface 2904 that are communicatively coupled via a local interface. The local interface can be, for example, but not limited to, one or more buses or other wired or wireless connections, as is known in the art. The local interface may have additional elements, which are omitted for simplicity, such as controllers, buffers (caches), drivers, repeaters, and receivers, to enable communications. Further, the local interface may include address, control, and/or data connections to enable appropriate communications among the other computer components.

Processor/controller is a hardware device for executing software, particularly software stored in memory. Processor can be any custom made or commercially available processor, a central processing unit (CPU), an auxiliary processor among several processors associated with the external controller/charger 2902 and/or control board 3000, a semiconductor based microprocessor (in the form of a microchip or chip set or other microprocessor, or generally any device for executing software instructions. Examples of suitable commercially available microprocessors are as follows: a PA-RISC series microprocessor from Hewlett-Packard Company, an 80×86 or Pentium series microprocessor from Intel Corporation, a PowerPC microprocessor from IBM, a Sparc microprocessor from Sun Microsystems, Inc., STMicroelectronics' STM32 family of microprocessors, or a 68xxx series microprocessor from Motorola Corporation. Processor may also represent a distributed processing architecture such as, but not limited to, SQL, Smalltalk, APL, KLisp, Snobol, Developer 200, MUMPS/Magic.

Memory can include any one or a combination of volatile memory elements (e.g., random access memory (RAM, such as DRAM, SRAM, SDRAM, etc.)) and nonvolatile memory elements (e.g., ROM, hard drive, tape, CDROM, etc.). Moreover, memory may incorporate electronic, magnetic, optical, and/or other types of storage media. Memory can have a distributed architecture where various components are situated remote from one another, but are still accessed by processor.

The software in memory may include one or more separate programs. The separate programs comprise ordered listings of executable instructions for implementing logical functions. The software in memory includes a suitable operating system (O/S). A non-exhaustive list of examples of suitable commercially available operating systems is as follows: (a) a Windows operating system available from Microsoft Corporation; (b) a Netware operating system available from Novell, Inc.; (c) a Macintosh operating system available from Apple Computer, Inc.; (d) a UNIX operating system, which is available for purchase from many vendors, such as the Hewlett-Packard Company, Sun Microsystems, Inc., and AT&T Corporation; (e) a LINUX operating system, which is freeware that is readily available on the Internet; (f) a run time Vxworks operating system from WindRiver Systems, Inc.; or (g) an appliance-based operating system, such as that implemented in handheld computers or personal digital assistants (PDAs) (e.g., PalmOS available from Palm Computing, Inc., and Windows CE available from Microsoft Corporation). Operating system essentially controls the execution of other computer programs and provides scheduling, input-output control, file and data management, memory management, and communication control and related services.

Steps and/or elements, and/or portions thereof of the present invention may be implemented using a source program, executable program (object code), script, or any other entity comprising a set of instructions to be performed. With a source program, the program needs to be translated via a compiler, assembler, interpreter, or the like, which may or may not be included within the memory, so as to operate properly in connection with the O/S. Furthermore, the software embodying the present invention can be written as (a) an object oriented programming language, which has classes of data and methods, or (b) a procedural programming language, which has routines, subroutines, and/or functions, for example but not limited to, C, C++, Pascal, Basic, Fortran, Cobol, Perl, Java, Arduino and Ada.

The I/O devices may include input devices, for example but not limited to, the differential pressure sensor 3010, the air pressure sensor 3030, the power/control terminal 3050, other input modules for PLCs, a keyboard, mouse, scanner, microphone, touch screens, interfaces such as bar code readers, stylus, laser readers, radio-frequency device readers, etc.

Examples of some of these devices can be found at (1) https://sensirion.com/products/catalog/SDP31); (2) https://www.digikey.com/en/products/detail/honeywell-sensing-and-productivity-solutions/MPRLS0025PA00001A/7915759? gclsrc=aw.ds&gad_source=1&gad_campaignid=20232005509&gbraid=0AAAAADrbLlj_7K ISiX1F0C0IWPxEv-uhB&gclid=Cj0KCQjw-ZHEBhCxARIsAGGN96KVB5931xT6lP0; and (3) https://www.digikey.com/en/products/detail/nxp-usa-inc/MPXM2102AS/420523? gclsrc=aw.ds&gad_source=1&gad_campaignid=20632149052&gbraid=0AAAAADrbLliUb Y95ut2H8MJvTEuH1fbrz&gclid=Cj0KCQjw-ZHEBhCxARIsAGGN96IrAgCaZ_QcmWaaAvyBRtuyXTtbxQ5VRTdLFN1Iu69SXUQpplq Fsw8a.

Furthermore, the I/O devices may also include output devices, for example but not limited to, the touch screen control display 2904, output modules for PLCs, a printer, bar code printers, displays, etc. Finally, the I/O devices may further include devices that communicate both inputs and outputs, for instance but not limited to, a modulator/demodulator (modem; for accessing another device, system, or network), a WiFi interface, a radio frequency (RF) or other transceiver, a telephonic interface, a bridge, and a router, and an information/power cable for connecting the control board to the external controller/charger 2902.

The software in the memory may further include a basic input output system (BIOS) as may be needed for certain controllers. The BIOS is a set of essential software routines that initialize and test hardware at startup, start the O/S, and support the transfer of data among the hardware devices. The BIOS is stored in ROM so that the BIOS can be executed when the controllers are activated.

When controllers are in operation, the processor is configured to execute software stored within memory, to communicate data to and from memory, and to generally control operations of controllers pursuant to the software. The present invention and the O/S, in whole or in part, but typically the latter, are read by processor, perhaps buffered within the processor, and then executed.

When the present invention is implemented in software, it should be noted that the software can be stored on any computer readable medium for use by or in connection with any computer related system or method. In the context of this document, a computer readable medium is an electronic, magnetic, optical, or other physical device or means that can contain or store a computer program for use by or in connection with a computer related system or method. The control and other operations of present invention can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the instruction execution system, apparatus, or device. The computer readable medium can be for example, but not limited to, an electronic, magnetic, optical, electromagnetic, infrared, or semiconductor system, apparatus, device, or propagation medium. More specific examples (a non-exhaustive list) of the computer-readable medium would include the following: an electrical connection (electronic) having one or more wires, a portable computer diskette (magnetic), a random access memory (RAM) (electronic), a read-only memory (ROM) (electronic), an erasable programmable read-only memory (EPROM, EEPROM, or Flash memory) (electronic), an optical fiber (optical), and a portable compact disc read-only memory (CDROM) (optical).

For that purpose, at least the external controller/charger 2902 can be equipped with network communication equipment and circuitry. In a preferred embodiment, the network communication equipment includes a wireless interface, such as WiFi or BLUTOOTH.

The positive airflow apparatus 2, 2′ and controllers 3020, 2902, and the software/firmware therein, are configured to transmit information to, and receive information from an App and/or remote computer application via the Internet or other network, for at least downloading new software/firmware to the apparatus 2, 2′ and setting up and directing the settings/operation of the controllers and software/firmware therein.

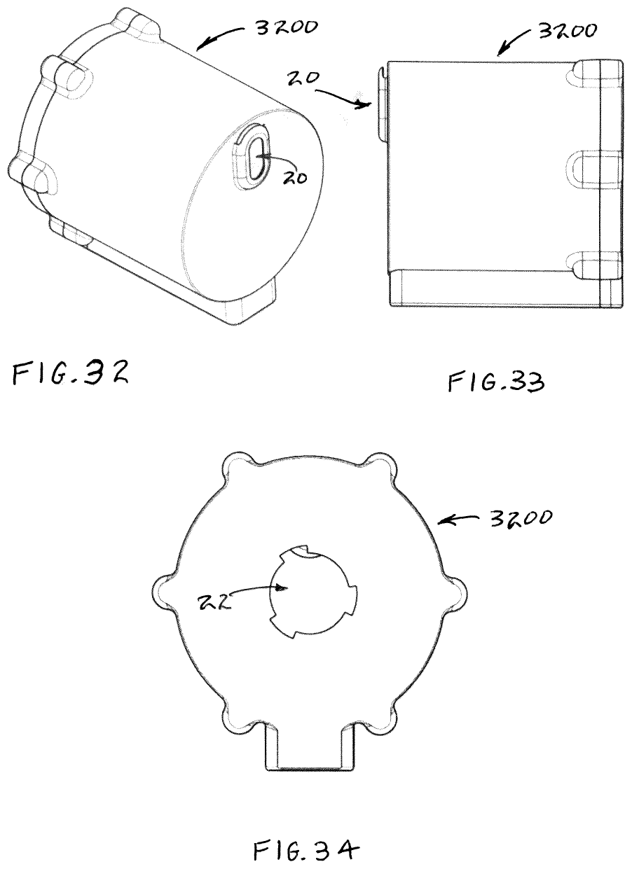

Referring to FIGS. 32-34, a silicone wrapped motor housing or wrapping 3200 is shown, which can wrap around the motor housing 10, 400, 400′, including the interior components of the motor housing 10, 400, 400′.

Any process descriptions or blocks in figures represented in the figures should be understood as representing modules, segments, portions of code, or electronics which include one or more executable instructions for implementing specific logical functions or steps in the process, and alternate implementations are included within the scope of the embodiments of the present invention in which functions may be executed out of order from that shown or discussed, including substantially concurrently or in reverse order, depending on the functionality involved, as would be understood by those having ordinary skill in the art.

While the specific embodiments have been illustrated and described, numerous modifications come to mind without significantly departing from the spirit of the invention, and the scope of protection is only limited by the scope of the accompanying Claims.

What is claimed is:

1. A positive airflow pressure apparatus for generating a positive airflow pressure in a user's breathing air passage, comprising:
a motor housing having a housing inlet and a housing outlet;
a motor housed within the motor housing for receiving air from the housing inlet and for propelling the air to the housing outlet, for creating positive airflow pressure at the housing outlet;
a face mask for breathing engagement with the user's air breathing passage, the face mask attached to the motor housing proximate the housing outlet, for receiving the propelled air through the housing outlet, wherein the motor and the face mask are for operating together to generate the positive airflow pressure in the user's breathing air passage; and,
a face mask connector for physically attaching the face mask and the motor housing to the user.

2. The positive airflow pressure apparatus of claim 1, wherein the motor housing comprises a main housing portion, an end cap, and an inlet plate.

3. The positive airflow pressure apparatus of claim 1 further comprising a first sealing gasket and second sealing gasket, the first sealing gasket circumferentially surrounding an outlet portion of the motor and the second sealing gasket circumferentially surrounding an inlet portion of the motor, the first sealing gasket and the second sealing gasket are positioned adjacent one another proximate a central portion of the motor.

4. The positive airflow pressure apparatus of claim 1, wherein the face mask comprises:
a main mask portion for covering at least one of a user's breathing air passageways;
a connection receiver for receiving and attaching the face mask connector to the face mask, for physically attaching the face mask and the motor housing to the user.

5. The positive airflow pressure apparatus of claim 4, wherein the connection receiver comprises:
a first connection receiver for receiving and removably attaching a left portion of a bottom connector of the face mask connector to the face mask;
a second connection receiver for receiving and removably attaching a right portion of the bottom connector of the face mask connector to the face mask;
a third connection receiver for receiving and attaching a top connector of the face mask connector to the face mask;

6. The positive airflow pressure apparatus of claim 5, wherein the first connection receiver is attached to a left side of the main mask portion, wherein the second connection receiver is attached to a right side of the main mask portion, and wherein the third connection receiver is attached to a front top portion of the main mask portion via a support.

7. The positive airflow pressure apparatus of claim 6, wherein the third connection receiver comprises:
a T-joint extending from the support;
a first arm extending from the left side of the T-joint; and,
a second arm extending from the right side of the T-joint.

8. The positive airflow pressure apparatus of claim 7, wherein the first arm is for receiving and removably attaching a left portion of a top connector of the face mask connector to the face mask, and the second arm is for receiving and removably attaching a right portion of the top connector of the face mask connector to the face mask.

9. The positive airflow pressure apparatus of claim 4, wherein the main mask portion of the face mask further comprises air holes for allowing air to exit the face mask when the user exhales.

10. The positive airflow pressure apparatus of claim 1, wherein the face mask further comprises:
an air treatment chamber side wall extending from a front portion of the face mask; and,
an air treatment chamber front wall extending from the air treatment chamber side wall to create an air treatment chamber for receiving an air treatment device.

11. The positive airflow pressure apparatus of claim 1, wherein the face mask further comprises a housing connector attached to a front portion of the face mask, and wherein the housing connector includes a flat front wall and a plurality of tabs for removably connecting the face mask to the motor housing.

12. The positive airflow pressure apparatus of claim 1, wherein the face mask further comprises a housing connector attached to a front portion of the face mask for removably connecting the face mask to the motor housing, and wherein a bore is formed through a central portion the housing connector for allowing air to flow between the housing connector and the motor housing.

13. The positive airflow pressure apparatus of claim 1, wherein the face mask further comprises an air restrictor attached to a front portion of the face mask for allowing air to enter the face mask from the motor housing, and for preventing air from flowing back into the motor housing from the face mask.

14. The positive airflow pressure apparatus of claim 1, wherein the motor housing comprises a main housing portion and an end cap.

15. The positive airflow pressure apparatus of claim 14, wherein the end cap comprises an interior side wall establishing an air through hole, wherein a plurality of flanges extend from the interior side wall for receiving a respective plurality of tabs of the face mask, and for allowing for removable attachment of the motor housing to the face mask.

16. The positive airflow pressure apparatus of claim 1, wherein the face mask connector comprises:
a top connector comprising a top elongated portion, a left closed end loop, and a right closed end loop;
a bottom connector comprising a bottom elongated portion, a left open end loop, and a right open end loop;
a joiner for connecting the top elongated portion of the top connector to the bottom elongated portion of the bottom connector, for maintaining generally consistent distance between the top elongated portion and the bottom elongated portion.

17. The positive airflow pressure apparatus of claim 16, wherein the bottom connector further comprises a plurality of left through holes and a plurality of right through holes;
wherein the left open end loop comprises a left end dowel;
wherein the right open end loop comprises a right end dowel;
wherein the left end dowel is for insertion in at least one of the plurality of left through holes for securing and connecting the left open end loop to a first connection receiver of the face mask;
wherein the right end dowel is for insertion in at least one of the plurality of right through holes for securing and connecting the right open end loop to a second connection receiver of the face mask; and,
wherein the left closed end loop and the right closed end loop are for securing and connecting the top connector to a third connection receiver of the face mask.

18. The positive airflow pressure apparatus of claim 17, wherein the bottom connector further comprises a left hook have a left opening and a right hook having a right opening, wherein the left open end loop is inserted within the left opening and the right open end loop is inserted in the left opening, wherein the left hook is for securing and connecting the left open end loop to the first connection receiver of the face mask, and wherein the right hook is for securing and connecting the right open end loop to the second connection receiver of the face mask.

19. The positive airflow pressure apparatus of claim 2, wherein the inlet plate comprises the housing inlet, wherein the inlet plate is in sealed engagement with the main housing portion and defining a housing front end chamber; wherein the motor is a variable speed motor; and,
wherein the positive airflow pressure apparatus further comprises:
an air restrictor plate secured in the housing front end chamber for separating the housing front end chamber into a first front end chamber on a first side of the air restrictor plate and a second front end chamber on a second side of the air restrictor plate, the air restrictor plate having restrictor air holes;
a differential pressure sensor positioned between the first front end chamber and the second front end chamber for sensing a pressure differential between the first front end chamber and the second front end chamber;
an air pressure sensor for sensing an air pressure proximate the housing outlet; and,
an onboard controller for receiving a pressure differential signal representing the pressure differential between the first front end chamber and the second front end chamber;
for receiving an air pressure signal representing the air pressure proximate the housing outlet;
and for continuously determining and controlling the speed of the variable speed motor in response to the sensed air pressure and the sensed pressure differential, for generating the positive airflow pressure.

20. The positive airflow pressure apparatus of claim 19, further comprising at least one of a rechargeable battery and/or a wired power module, connected to and powering the onboard controller and motor.
